(19) United States
(12) Patent Application Publication
(10) Pub. No.: US 2015/0088431 A1
(43) Pub. Date: Mar. 26, 2015

(54) DYNAMIC PROFILES

(71) Applicant: Leo Technologies, Inc., Boulder, CO (US)

(72) Inventors: Ronald Podhajsky, Boulder, CO (US); Arlen J. Reschke, Longmont, CO (US)

(21) Appl. No.: 14/491,878

(22) Filed: Sep. 19, 2014

Related U.S. Application Data
(60) Provisional application No. 62/027,079, filed on Jul. 21, 2014.
(60) Provisional application No. 61/943,997, filed on Feb. 24, 2014.
(60) Provisional application No. 61/880,872, filed on Sep. 21, 2013.
(60) Provisional application No. 61/880,868, filed on Sep. 21, 2013.

(51) Int. Cl. G06F 19/10; G01N 21/59; G01N 21/84; A61B 5/0205; A61B 5/00

(52) U.S. Cl. CPC G06F 19/10; A61B 5/0205; A61B 5/0059; A61B 5/443; G01N 21/84; G01N 21/59; G01N 2201/062
USPC 702/19; 600/483; 600/479

(57) ABSTRACT

Implementations disclosed herein provide a monitoring technology. In one implementation, a monitoring system measures whole body biometric levels by analysis of changes in vascular volume caused by pulsatile pressure waves and in tissue volume in response to the pulsatile pressure. The monitoring system includes a monitoring device, which uses a light-based measurement technique to measure biometric levels during different activities and at rest. A light source operatively connected to a light sensor, transmits light, reflectively or transmissively, through tissue. The light sensor detects absorption of the light. Based on wavelength measurements of the detected light, the monitoring device produces a PPG waveform representing characteristic effects of certain physiological parameters. In one implementation, operating contexts are sensed in a monitoring device. A monitoring profile is selected based on the sensed operating contexts. A biometric is computed based on the PPG waveform and on the selected monitoring profile.

### CROSS REFERENCE TO RELATED APPLICATIONS

The present application claims priority to pending U.S. Provisional Patent Application Ser. No. 61/880,868, entitled “System and Method for Monitoring Body Hydration Levels with a Non-Obtrusive Form Factor,” filed on Sep. 21, 2013, U.S. Provisional Patent Application Ser. No. 61/880,872, entitled “System and Method for Non-Invasive Plethysmogram Measurement,” filed on Sep. 21, 2013, U.S. Provisional Patent Application No. 61/943,997, entitled “Algorithm that Derives Hydration Levels From a Plethysmogram,” filed on Feb. 24, 2014, and U.S. Provisional Patent Application Ser. No. 62/027,079, entitled “Hydration Monitoring,” filed on Jul. 21, 2014, all of which are specifically incorporated by reference for all they disclose and teach.

The present application is related to U.S. patent application Ser. No. ______ [Docket No. 277002USP2], entitled “Measuring Tissue Volume With Dynamic Autoreconfiguration,” U.S. patent application Ser. No. ______ [Docket No. 277002USP1], entitled “Hydration Monitoring,” and U.S. patent application Ser. No. ______ [Docket No. 277003USP1, entitled “Data Integrity,” all of which are filed concurrently herewith, and specifically incorporated by reference for all they disclose and teach.

### BACKGROUND

Physiological characteristics in the body, including hydration, can be measured by a variety of techniques, such as skin electrical impedance or optical spectroscopic techniques. Optical spectroscopic techniques may include detecting a photoplethysmographic (PPG) waveform using optical transmitters and optical sensors. In some implementations, PPG signals measure local blood pressure changes in a user's extremity or by ventilation. These waveform measurements can then be analyzed for assessing certain biological conditions.

### SUMMARY

Implementations disclosed herein provide a hydration monitoring technology, although other biometrics may also be determined using or in combination with other similar techniques. In one implementation, a hydration monitoring system measures whole body hydration levels by analysis of changes in vascular volume caused by pulsatile pressure waves and in tissue volume in response to the pulsatile pressure. The hydration monitoring system includes a hydration monitoring device, which uses a light-based measurement technique to measure hydration levels and heart rate during different activities and at rest. In one implementation, a light source operatively connected to a light sensor, transmits light, reflectively or transmissively, through tissue. The light sensor detects absorption of the light. Based on wavelength measurements of the detected light, the hydration monitoring device generates a PPG waveform representing characteristic effects of hydration.

In one implementation, one or more operating contexts are sensed via one or more environmental sensors in a monitoring device. At least one monitoring profile of a set of monitoring profiles is selected based on the one or more sensed operating contexts. A biometric is computed based on data samples monitored by the monitoring device and on the selected at least one monitoring profile.

This Summary introduces a selection of concepts in a simplified form that are further described below in the Detailed Description. This Summary is not intended to identify key features or essential features of the claimed subject matter, nor is it intended to be used to limit the scope of the claimed subject matter. Other feature, details, utilities, and advantages of the claimed subject matter will be apparent from the following more particular Detailed Description of various implementations as further illustrated in the accompanying drawings and defined in the appended claims.

### BRIEF DESCRIPTIONS OF THE DRAWINGS

FIG. 1 illustrates an example hydration monitoring system.

FIG. 2 illustrates a block diagram of an example hydration monitoring system circuitry.

FIG. 3 graphically depicts an example plethysmograph in a hydration monitoring system.

FIG. 4 graphically depicts a second example plethysmograph in a hydration monitoring system.

FIG. 5 illustrates example operations for determining PPG pulse data sample integrity of hydration monitoring data.

FIG. 6 illustrates example operations for determining hydration metric data results integrity of hydration monitoring data.

FIG. 7 graphically depicts hydration metric data results.

FIG. 8 illustrates example operations for determining a dynamic profile with hydration monitoring data.

FIG. 9 illustrates a block diagram of an example computer system used to implant a hydration monitoring system.

### DETAILED DESCRIPTION

Devices, methods, and software using sensors and light sources may be used to produce PPG waveform measurement of hydration levels and heart rate during different activities and at rest. The disclosed technology provides whole body hydration levels by optically measuring changes in vascular volume caused by pulsatile pressure waves and responses by proximal tissue to the pulsatile pressure. Such measurements for whole body hydration levels can be made at a test region (e.g., a wrist). A hybrid of systemic and local hydration monitoring is achieved by measuring both vascular volume and tissue biomechanics that produces more accurate results, which can be communicated in a hydration metric.

In addition to hydration, in other implementations, the disclosed technology also monitors or refines results of monitoring other physiological parameters, including, but not limited to, blood pressure, heart contractility hydration, heart rate, heart contractility, valve performance, vascular compliance, baroreceptor engagement, systemic neural response, local neural response, vascular branch reflections, blood density, vascular pathology, valve pathology, heart pathology, and compensatory reserve index. The data of these other physiological parameters may be used to compute a biometric pursuant to the technology disclosed herein.

To calculate either a hydration metric or other biometric related data, a hybrid of changes in vascular and tissue pressures and/or volumes are analyzed using a light-based measurement technique. In one implementation, the system includes a processor in operative communication with an optical sensor or light sensor and a light source. The light source exposes tissue to light. Light can be reflected through the tissue, or the light can be transmitted through the tissue. The light sensor is configured to detect changes of light absorption through the body tissue to measure changes in body tissue volume in combination of changes in vascular volume within a test region of the body of a subject.

Absorption of a specific wavelength of light energy is dependent on the amount of oxygenated blood in the vessels. Since the heart is a pulsatile pump, blood enters the arteries intermittently with each heartbeat increasing vascular volume and/or pressure. Vessels expand and contract, in response to the changing pressure in the vessels. At the same time, pressure is also dependent on surrounding tissue, which may comprise as much as 60-80% water. When the vessels expand and relax, the amount of blood volume in the observed tissue increases and decreases. The compliance ability to distend and increase volume by pressure of the vessels changes in rhythm with the heartbeat. As overall tissue hydration increases, the compliance of the vessels, both centrally and peripherally, is reduced, and there is more resistance to pressure in the vasculature.

The light absorption in the tissue has a pulsatile component that varies in rhythm with the heartbeat. As the heart beats, the volume of blood increases and travels as a pressure wave through the circulatory system. As blood volume increases in the arteries, the received light intensity reduces. As blood volume in the arteries decreases, the light transmission increases.

A processor, operatively connected to the light sensor, processes the light changes in time variant signals (intensity vs. time) detected by a light sensor. The time variant signals can be amplified to generate an electrical representation in a measureable PPG waveform.

In another implementation, the plethysmographic waveform is measureable by non-optical means. For example, electrical impedance plethysmography also provides a waveform representing the changes in tissue volume and in vascular volume. For either optical or non-optical plethysmographic waveform generation, the measurement and computations of the disclosed technology remain the same.

The waveform provided by the photodetector may or may not be inverted. For illustration, if the waveform is inverted, the peak of the waveform corresponds to the maximum absorption of the light when the blood vessels are pulsing at their maximum dilation. The lowest part of the peak is the point between heartbeats where there is the minimum dilation of the vessels and less absorption of the light. The PPG waveform represents volume and pressure changes in the circulatory system indicative of characteristic effects of hydration.

Areas of the PPG waveforms are computed that represent the volume and pressure changes in the body. The first area of the PPG waveform, indicative of changes in tissue volume, is referred to as the “Tissue Pressure Area” or “TPA.” A second area of the PPG waveform, indicative of the changes in vascular volume, is referred to as the “Vessel Pressure Area” or “VPA.”

Based on these computations of areas, a hydration metric can be computed based on the different ratios of determined changes in vascular volume and tissue volume in the body. For example, during times of exercise or following exertion, the ratio of the TPA to the VPA provides a hydration metric:

$$HydrationIndex = \frac{TissuePressureArea}{VesselPressureArea}$$

Alternatively, during extended times of rest, the ratio of the VPA to the TPA provides a hydration metric:

$$HydrationIndex = \frac{VesselPressureArea}{TissuePressureArea}$$

These ratios can be inverted and can vary subject to change depending on a variety of factors, including the level of activity (e.g., rest, during exercise, following exertion), physiological conditions, environmental conditions, particular user profile parameters, the specific hydration monitoring device used, or calibration of the hydration monitoring system.

For example, in one implementation, during rest, the hydration index can be computed correlating to a ratio of the TPA to the VPA. Or, in another implementation, during exercise, the hydration index can be computed correlating to a ratio of the VPA to the TPA. Or in another implementation, a particular user profile may trigger a change in taking the ratio of the VPA to the TPA, to taking a ratio of the TPA of the VPA.

In another implementation, multipliers or constants may be used to calculate the hydration metric with the ratio of the TPA to the VPA or the ratio of the VPA to the TPA. Such modification of the ratio can result in better aggregate data. These multipliers or constants can also be implemented as part of a user profile.

Once the hydration metric is computed, the hydration monitoring system communicates the hydration metric for presentation via a user interface.

The technology disclosed herein includes devices, methods, and software for selecting PPG pulse data samples and hydration metric data results that satisfy data integrity conditions and result integrity conditions for such described hydration monitoring technology. In some implementations, PPG pulse data samples may be selected and/or filtered by monitoring profiles based on one or more operating contexts (e.g., an environmental condition, a sensed activity, or a physiological condition).

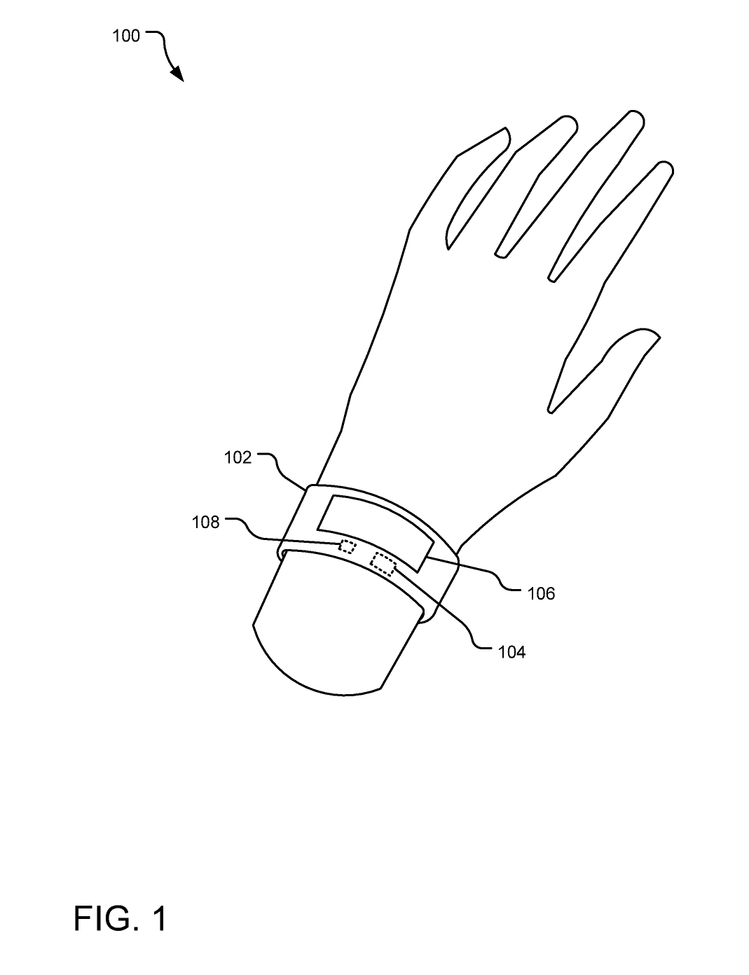

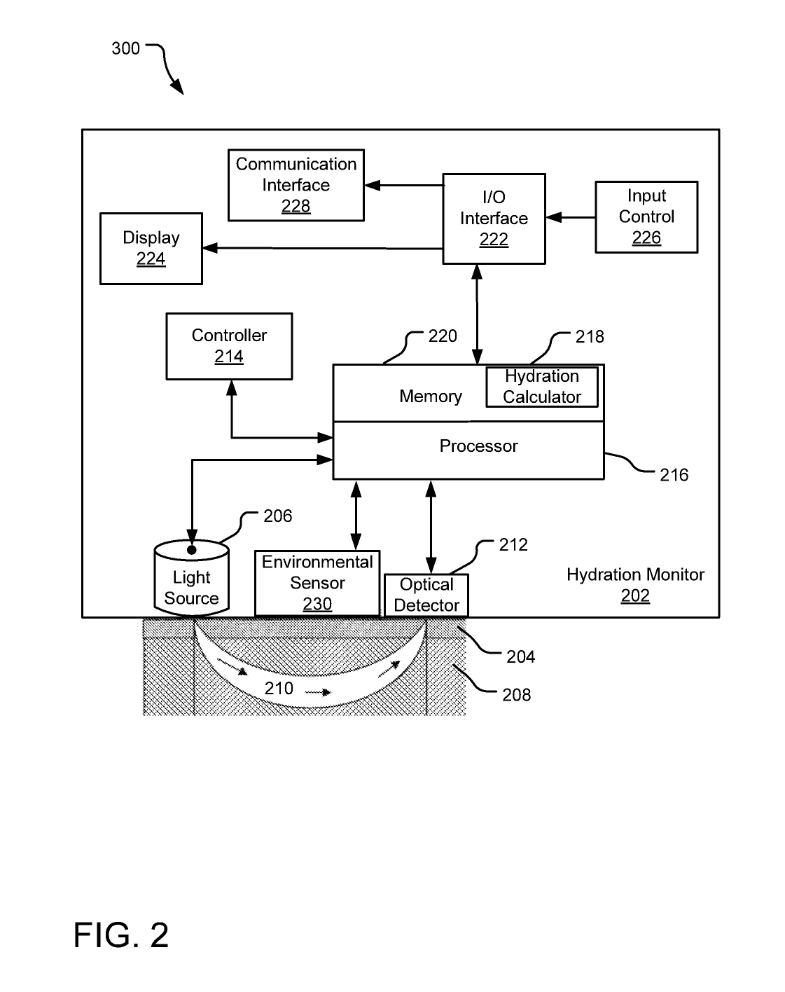

In FIG. 1, an example hydration monitoring system 100 in the disclosed technology is shown. The system 100 includes sensor circuitry (described further in FIG. 2) configured to acquire and reflectively measure a PPG waveform. The sensor circuitry may be located in a device or monitor, such as a wrist-worn form factor (e.g., watch or wristlet 102), as shown in FIG. 1. Other implementations may include transmissive PPG measurement systems worn on the fingertip, earlobe, etc., or reflective PPG systems worn on the forehead, fingertip, or other body locations.

Other implementations may include a PPG waveform sensor module that may be incorporated into expandable bandages, clothing (e.g. sweatbands, gloves, sports bras, and other sportswear), sports equipment (e.g., a bike helmet), ear buds, or an anklet Additional implementations may include the sensor module incorporated into an accessory housing or protective cover used with smart phones, tablets, GPS, and other similar devices. In another implementation, the sensor may be incorporated into a switch button used on a monitoring device or may be incorporated as a biometric contact button exclusively for biometric data readings. In another implementation, monitoring may be facilitated through the device itself, a monitoring service, a computer, wirelessly, or via a medical testing unit.

The PPG waveform sensor module may also be incorporated as a biometric button, such as a finger or a palm contact location. The module may also be incorporated into health and fitness equipment, such as treadmills, elliptical trainers, bicycle handlebars, water bottles, and other similar equipment.

Referring to FIG. 1, the wristlet 102 has a light detector or light sensor 104 and a light source 108. The light sensor 104 and the light source 108 can be configured to rest on or next to the skin surface in close proximity to the arterial or arteriole vascular components that produce a PPG wave. As further described in detail in FIG. 2, the light source 108 generates light through skin and tissue, and the light is detected by the light sensor 104. A processing unit in the wristlet 102 (or accessible to the wristlet 102) processes the light into analytical PPG pulse data samples, which are then processed into hydration metric data results. The hydration metric data results are displayed on an interface or display 106.

The light sensor 104 and the light source 108 can be located in various configurations and locations in the hydration monitoring system 100. In FIG. 1, a light sensor 104 is located on the inside of the wristlet 102, adjacent to the user's skin. In another implementation (not shown), the light sensor 104 and the light source 108 may be located on the side of the wristlet 102. In this example, a user can wear the wristlet on one wrist, and use the wristlet for measurement in the other wrist or finger on the other arm. In another implementation (not shown), a sensor could be on the top of a wristlet 102, wherein the sensor detects hydration in a person other than the person wearing the wristlet 102 (e.g., a patient uses a first responder's watch to read their hydration). In another implementation (not shown), a wristlet may have a light sensor positioned on one side of the wristlet aimed into the wrist, and another light sensor may be located on another side of the wristlet.

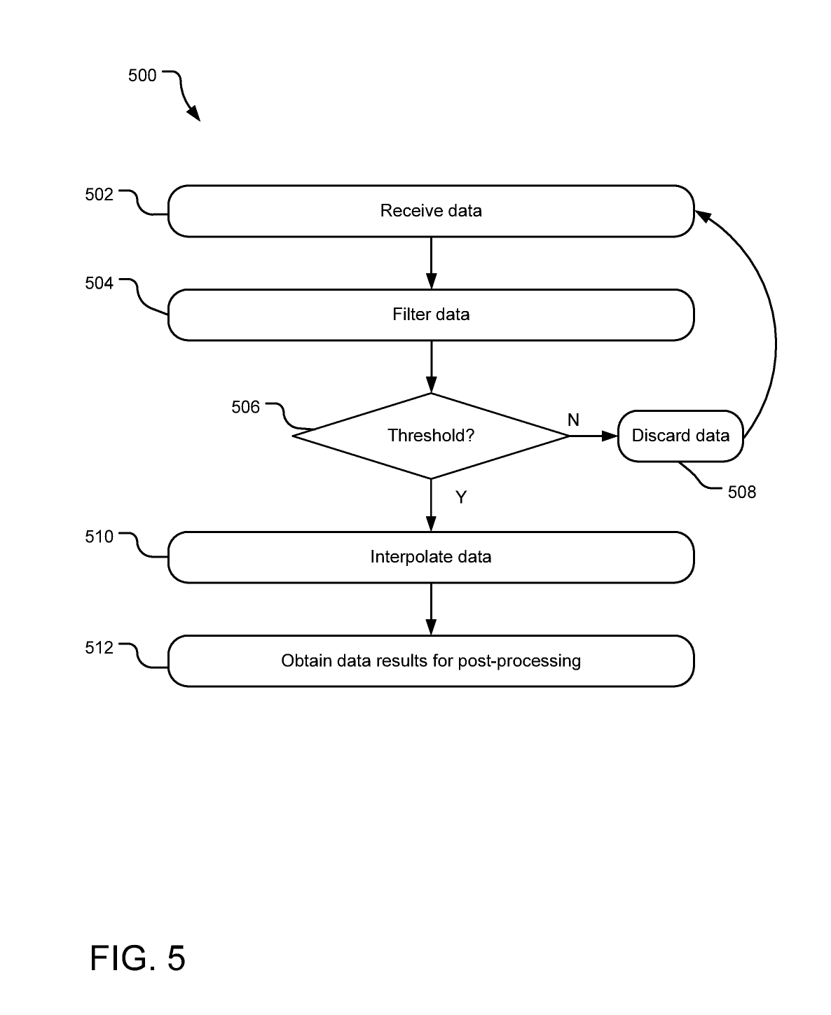

In another implementation (not shown), there can be a plurality of light sources 108 and a plurality of light sensors 104 configured in an array. There may be an array of light sensors 104 and light sources 108 (e.g., LEDs), which can be configured to rest on or next to the skin surface around the wrist. The array may be configured to select an optimal pairing of the light sensors and light sources that provides the best representation of the PPG waveform (described in more detail in FIG. 5).

In another implementation (not shown), there may be a plurality of LEDs, wherein one LED may be a light source and another LED may be a sensor. In another implementation (not shown), the light sensor 104 may be a near infrared spectrometer and the light source 108 may provide light in the near infrared wavelength. In another implementation (not shown), where there is sufficient ambient light, the hydration monitoring system consists of using only a photodetector or other optical sensor.

In another implementation, electrodes (e.g., conductive ground pins) are located on the interior of the wristlet and configured to be in contact with the surface of a user's skin. The ground pins measure impedance or resistance. The hydration monitoring system can monitor for skin contact integrity and surface moisture. If there is inadequate skin contact, system modifications can be made. For example, an alarm may signal the user that there is inadequate contact, and the user can readjust the fitting of the wristlet.

FIG. 2 shows a block diagram of an example hydration monitoring system circuitry 200 that is configured to acquire and measure a PPG waveform and determine a hydration metric representative of hydration levels in the body, which can be revealed on a display connected to the monitor. As shown in FIG. 2, the processor performs these operations in one hydration monitor 202. However, in other implementations, the PPG waveform may be obtained from an external source and measured for computation of the hydration metric in a hydration monitoring system circuitry 200.

In the hydration monitoring system circuitry 200 in FIG. 2, a hydration monitoring circuitry operates to monitor hydration when a user places a hydration monitor 202 against external skin 204 (e.g., on a user's wrist). A controller 214 sends signals to a processor 216 to activate a light source (e.g., LED) 306. The light source 206 generates light 210 against a skin 204. The light 210 is reflected through the skin 204, through a tissue 208 and through the skin 204 again for collection by an optical detector or light sensor 212.

The light sensor 212 detects the PPG waveform as a varying voltage or current level that varies with time. The relationship of the varying voltage (or current level) of the PPG waveform may be dependent on time, and can be defined as a function, or as a relationship between two variables (voltage amplitude and time) such that to each value of the independent variable (time) there corresponds a value of the dependent variable (voltage amplitude).

The processor 216 operates as a hydration metric monitoring processor and determines changes in tissue hydration levels based on the detected changes in light 210. The processor 216 interpolates PPG pulse data samples, from the light sensor 212.

The processor 216 filters the PPG pulse data samples using profiles, which comprise thresholds, margins, and/or parameters based on contexts (e.g., motion, heart rate, temperature, and sweat volume). The profiles may be stored in a memory 220 or received from an external source. The processor 216 measures the filtered PPG pulse data samples and computes tissue pressure areas and vessel pressure areas, indicative of changes in tissue volume and changes in vascular volume, respectively.

A hydration calculator 218 is also stored in a memory 220 in the processor 216. The hydration calculator computes a ratio of the tissue pressure area to the vessel pressure area to obtain a hydration metric or other output representative of hydration level. Or, in another implementation, as provided above, the ratio may be inverted, and/or it may include multipliers or constants in an equation to compute the hydration metric. The hydration metric or other output value from the hydration calculator 218 may be input into an input/output (I/O) interface 222. The I/O interface 222 is connected to one or more user-interface devices (e.g., a display unit 224) and a communications interface 228.

In one implementation, the hydration metric can be displayed on a user-interface device or display unit 224. In another implementation, the hydration metric or other output value may be communicated to the communications interface 228 for purposes of sending a signal or alarm to the user via a device, a monitoring service, a computer, wirelessly, or via a medical monitoring unit. For example, if there is an output value indicating dehydration in a patient, a communications interface 228 may signal an alarm to the patient or medical staff via a device or medical monitoring unit.

The processor can process the PPG pulse data through various algorithms and transforms (e.g., FIR filter, IIR filter, first derivative, second derivative, Fast Fourier Transform (FFT), etc.). As an example, the initial data can be analyzed with an FFT and a secondary analysis can determine whether characteristic power shifts have occurred that are correlated to a change in hydration, heart rate, etc.

The system 200 can also include one or more environmental sensors 230 that operatively communicate with a processor 216. In one implementation, an environmental sensor may be a temperature monitor configured to monitor the temperature of the tissue. Depending on certain profiles (e.g., a profile specific to temperature) that may be stored in the memory 220, PPG pulse data samples may be filtered by monitoring profiles based on one or more operating contexts. For example, if a user only wants PPG pulse data measurements within a range of a normal human body temperature (e.g., 97.7°-99.5° F.), and this range is part of the profile, PPG pulse data samples taken at a temperature outside the 97.7°-99.5° F. range may be filtered out and omitted during analysis. As a result, based on the premise that a data reading taken when the body is not at a normal temperature may not be reliable data, the filtered PPG pulse data samples provide more accurate measurement of tissue hydration. In another implementation, an environmental sensor (e.g., electrode) detects surface contact, or lack thereof, for filtering and analysis of reliable data. In yet another implementation, the environmental sensor is an accelerometer, which detects motion.

An input control 226 may also be connected to the I/O 222. The input control 226 may be a button, a pressure sensor, an RF sensor, or even a touch screen. Various information may be input into the input control 226. For example, if a certain dynamic profile analysis is desired, a user may input such a request. In another example, a user may input a target hydration level into the input control 226. If a user inputs a minimum target hydration level, an alarm may be activated once a minimum value is reached, and a user may be notified visually or audibly by the monitor or another device connected directly or wirelessly. If a user inputs a maximum target hydration level, for example, a professional athlete conditioning their body for a target hydration level, a similar notification will occur. In yet another example, if a user wants to measure hydration for certain time periods or temperatures, an input control 226 could be used for such purpose. In some implementations, the operation blocks of the system 200 may be connected by a radio transmitter.

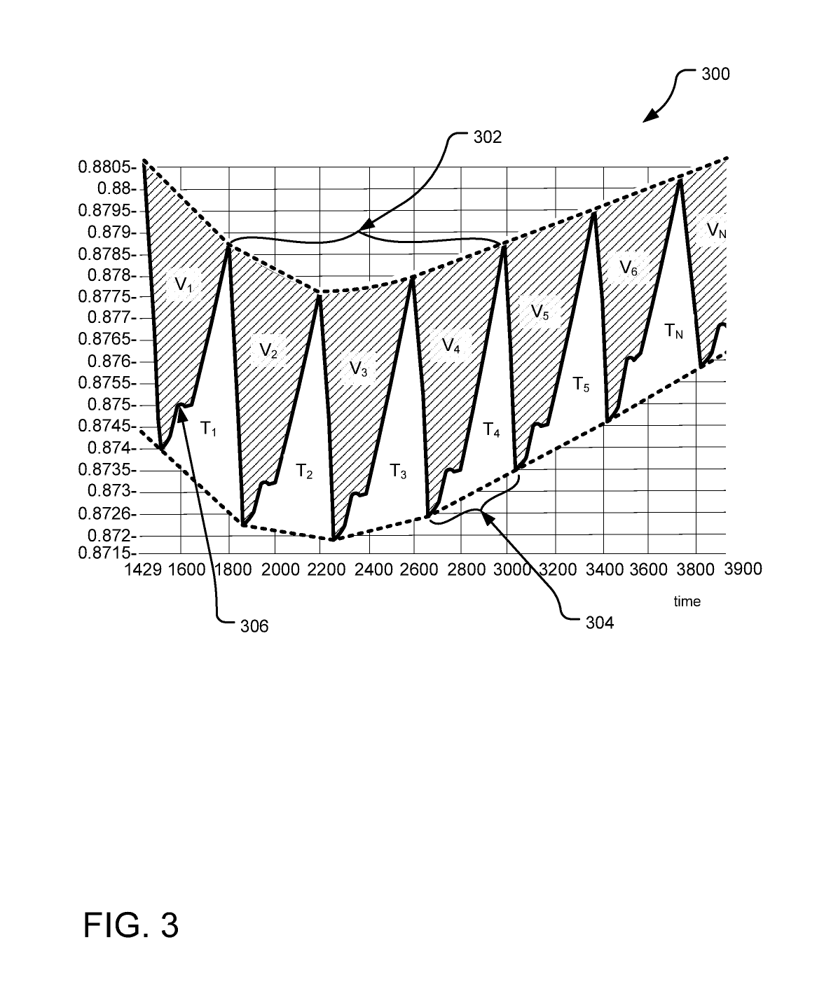

Referring to FIG. 3, an example plethysmograph 300 (measured in amplitude/time) in a hydration monitoring system graphically depicts a PPG waveform obtainable with the disclosed technology. As depicted graphically, when the heart contracts, pressure rises rapidly in the ventricle at the beginning of systole (beginning at approximately 0.8805) and soon exceeds that in the aorta. The aortic valve opens, blood is ejected, and aortic pressure rises. During the early part of the ejection, ventricular pressure exceeds aortic pressure. About halfway through ejection, the two pressures are the same and an adverse pressure gradient faces the heart (at approximately 0.874). The flow and pressure start to fall causing a “notch” in the aortic pressure wave (the dicrotic notch, shown in FIG. 3 as a dicrotic notch 306), also known as a reflected wave from the initial heart pulsatile wave. The dicrotic notch 306 marks the closure of the aortic valve. Thereafter, the ventricular pressure falls very rapidly as the heart muscle relaxes. The aortic pressure falls more slowly, with the aorta serving as a reservoir.

For illustrative purposes, the aorta may be considered as an elastic vessel or chamber and the peripheral blood vessels are considered as rigid tubes of constant resistance. For the elastic chamber (aorta), its change of volume is assumed to be absorbed by the compliance of the aortic walls as the aortic pressure increases. This elastic compliance of the aortic wall tends to smooth out the impulse of pressure the heart creates. Hence, the pressure wave as detected as a PPG waveform takes its characteristic shape.

The arterial branches that occur between the heart and the peripheral sensing site create reflection waves that also affect the shape of the PPG wave. The volume of blood has a direct effect on the PPG waveform as well as an effect on the peripheral and central nervous system, which responds in a way that affects the vessel compliance. This vessel compliance change is also reflected in the shape of the PPG wave. However, the simplifying assumption that the peripheral blood vessels are rigid tubes of constant resistance can be modified to encompass the changes that occur when tissue hydration is varying.

As overall tissue hydration increases, the compliance of the vessels, both centrally and peripherally, is reduced. This systemic reduction in vascular compliance due to systemic variance in tissue hydration can be detected as a shift in the shape of the PPG wave. The shift in shape of the PPG waveform may be detected in a way that is indicative of the relative change in tissue hydration level.

Prior to computation of a hydration metric, a PPG waveform data sample may be selected and/or filtered by monitoring profiles based on one or more sensed operating contexts sensed by an environmental sensor or one or more non-sensed operating contexts (e.g., demographic inputs, such as weight, gender, age, etc.). The monitoring profiles can select a data sample based on parameters in the monitoring profiles, including data sample satisfaction of data integrity or result integrity. The monitoring profiles are subject to change as operating contexts change. Further, computations (e.g., the ratio of changes of tissue volume and changes of vascular volume) are subject to change depending on a change in operating contexts and monitoring profiles.

In the selected PPG waveform, the locations and amplitudes of the local peaks of the PPG waveform are identified. Several methods may be used to find the minimum points and the maximum points of the PPG waveform. In one implementation, a method of a first-derivative test to locate the relative minimum and relative maximum points may be used on the PPG function. As shown in FIG. 3, the minimum points and the maximum points are traced within triangular-shaped tracing.

When the locations (“locs”) of the minimum points and the maximum points of the PPG waveform are identified, the heart rate may also be calculated using the following equation (in MatLab script):

$$HeartRate = ( \frac{100}{mean ( diff ( locs ) )} ) \cdot 60$$

In this equation, a value of 100 is used because a sample rate may be set at 100 samples per second. The term “diff(locs)” refers to the distance between each adjacent location. The mean of the distances is determined by “mean(diff(locs)) and the fraction is multiplied by 60 to convert the dimension from inverse seconds to “per minute.” The unit of the calculated heart rate is in beats per minute (bpm).

Once the locations and the amplitudes of the minimum points and the maximum points of the PPG waveform are identified, any two adjacent minimum points (or maximum points) serve to define a line connecting the two adjacent minimum points (or maximum points), which can be calculated using line equations.

In the PPG waveform orientation shown in FIG. 3, a line 302 connects the local maximum points represent the diastolic pressure of the test subject. A line 304 connects the local minimum points represent the systolic pressure of the test subject. It is very common in the medical field to invert the PPG waveform prior to displaying it. Many medical devices that display the PPG waveform inverted the waveform so that the blood pressure is increasing in the graph when the PPG curve is shown going up. This disclosure includes either orientation of the PPG waveform. All of the data analytics, calculations, and data manipulations disclosed herein apply to the PPG waveform whether the waveform is inverted or not inverted.

Using the lines 302 and 304, the areas between the curves in the PPG waveform can be defined. The area between the PPG curve and the diastolic curve may be defined as the “Vessel Pressure Area” or “VPA.” The VPA is filled with lines and is labeled V1, V2, V3, . . . , VN. The area between the systolic curve and the PPG curve is defined as the “Tissue Pressure Area” or “TPA.” The TPA is not filled with lines and is labeled T1, T2, T3, . . . , TN.

Several methods of calculating the area of a region between two curves may be used. In some implementations, the application of definite integrals from the area of regions under two different curves may be used. The process of calculating the area of a region between the two different curves or functions is to subtract the function with the lesser-valued area from the function with the greater valued area. This calculation then results in the calculated area between the two curves or functions. In another implementation, one function may be subtracted from the other prior to the process of integration.

Several methods of analyzing a definite integral by partitioning the area under a curve into sub-regions may also be used. The sub-regions are approximated by rectangles of know dimension so the areas of all the rectangles can be summated to approximate the area of the definite integral. If trapezoids are used instead of rectangles, the approximation is more accurate. The digitization of an analog biometric signal may be useful for this type of trapezoidal integration. An example of trapezoidal integration use in the hydration metric MatLab script that provides the area between the TPA and the VPA is calculated with the following equations:

TPA=trapz(PlethWave)−trapz(slocs,−spks)

VPA=trapz(dlocs,dpks)−trapz(PlethWave)

After a TPA and the VPA are derived from the PPG waveform, a hydration metric is derived correlating to a ratio of the TPA divided by the VPA (or correlating to a ratio of the VPA divided by the TPA, and/or with multipliers or constants, as provided above).

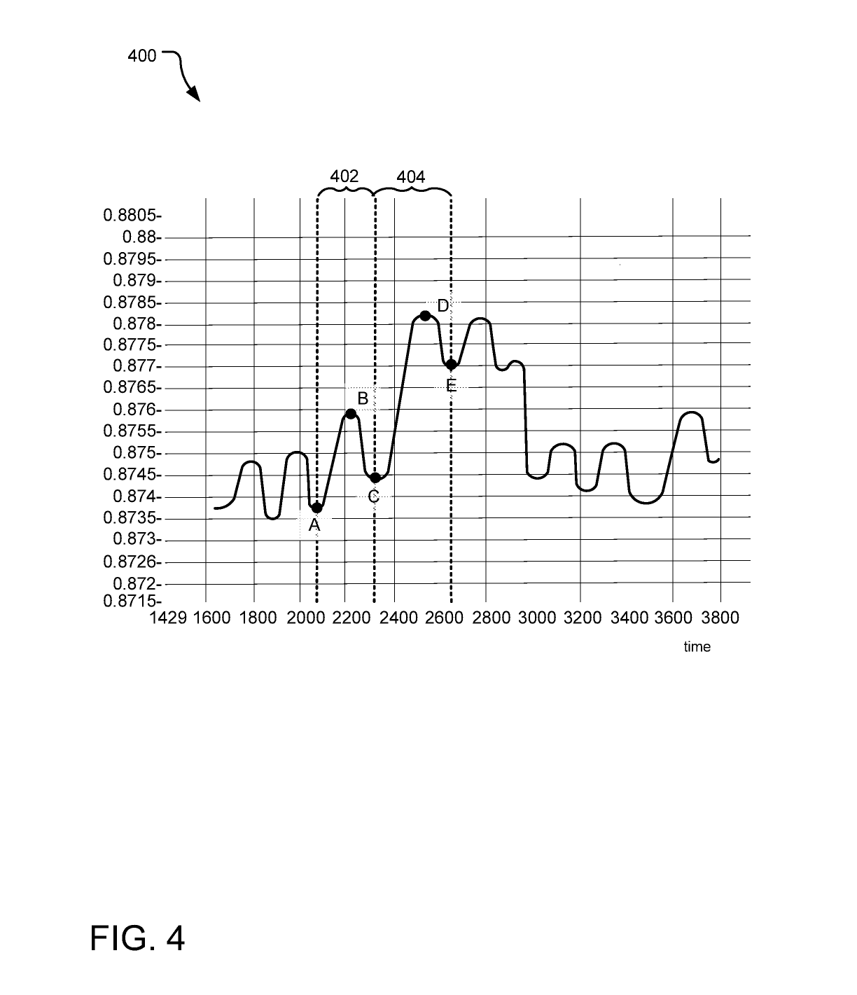

In FIG. 4, an example plethysmograph in a hydration monitoring system 400 is shown. The technology disclosed herein includes methods of differentiating between well-formed pulses in the PPG waveform and ones that have distortion in an effort to obtain accurate PPG pulse data samples for use in calculating the hydration metric. As illustrated, there may be a pulse 402 in the waveform, wherein a distance from a point A to a point B is measured (from minimum to maximum), and a distance from a point B to a point C is measured (from maximum to minimum). Next, a ratio of the distances from a point A to a point B and from a point B to a point C is taken. If the distances are relatively the same, then it may be determined that the PPG pulse data is most likely acceptable as a legitimate waveform. As shown in FIG. 4, the two distances in the pulse 402 are relatively the same and may likely be acceptable PPG pulse data samples.

If the ratio of distances across multiple pulses is smaller to larger, such change maybe indicative of invalid PPG pulse data samples. As shown in a pulse 404 in FIG. 4, there may a distortion in the PPG pulse data samples. The distance from a point C to a point D (from minimum to maximum), and a distance from a point D to a point E (maximum to minimum) in pulse 404, varies significantly. Taking a ratio of these two distances will reveal a large disparity, indicating unacceptable PPG pulse data samples. After determination that the PPG pulse data samples are unacceptable, the unacceptable PPG pulse data samples may be discarded or unused.

Several parameters may be responsible for such distortion resulting in unacceptable PPG pulse data samples. For example, sensing a PPG wave may be especially problematic when a user of a monitor is in motion. Distortion may occur when a person sneezes, laughs, or moves an extremity, causing the signal to fluctuate. In another example, unreliable waveforms may result if LED contact with the body changes. In other circumstances, sweat, or other physical, conditions of the user may also interfere with an accurate read.

In another implementation indicating potential invalid data, a drift in the waveform may be observed. If when taking the ratio of one distance in a pulse, and a second distance in a pulse, a signal is drifting, this may indicate that the user is standing up and the body is equalizing. The waveform with multiple peaks only become flat when the body nears equilibrium. If a person laughs or sneezes, for example, such movement can cause a momentary glitch or a pressure equalizing that causes drift. Therefore, by observing drift in the waveform, unacceptable PPG pulse data samples may be discarded. Such schemes are important, especially in the wrist for example, where there is a lot of artifactual information that may be invalid PPG pulse data samples.

FIG. 5 illustrates example operations for determining data integrity of hydration monitoring data before a hydration metric is calculated. Raw PPG pulse data samples may be received as a sequence of data samples from a sensor in a hydration device in a receiving operation 502. The PPG pulse data samples may be filtered against each other using a variety of pre-processing schemes for determining the integrity of data samples in a filtering operation 504. Such filtering schemes may use set conditions, such as amplitude, time, pulse recognition, and other shape parameters. For example, detection of erratic pulsatile behavior compared to normal behavior or and heart pumping action deviations can be analyzed. By measuring more than, for example, a three-point analysis used in pulse oximetry, the disclosed technology can obtain more accurate results and discard inaccurate results. In one embodiment, the discarded section of the PPG waveform is whole single pulse or multiples of single pulses that are identified by the three-point analysis or do not meet other set criteria. The gap created by discarding pulses is filled by uniting the acceptable pulses surrounding the discarded pulses.

One filtering scheme that may be used in filtering operation 504 to obtain acceptable PPG pulse data samples may include calculating heart rate (as described in FIG. 3). An early heart rate detection algorithm sets up the parameters to detect the peaks. The heart rate detection can refine and preload peak detection variables, dynamically.

In another implementation, a ratio of adjacent amplitudes per pulse may be taken to determine acceptable PPG pulse data samples. For example, referring to FIG. 4, this method would include taking adjacent amplitudes illustrated in the ratio of the distance between a point A to a point B, and the distance between a point B and a point C. If an average of pulses is taken, and one pulse is outside the average, the PPG pulse data samples may be ignored, or the value may be capped.

In another implementation, a time distance ratio filtering scheme may be used. This method includes measuring, for example, the ratio of the time between a point A to a point B, and the time between a point B and a point C, in FIG. 4.

In another implementation, a filtering scheme may include measuring the delta in absolute pulse height. This scheme is performed, for example, by taking the ratio of the pulse 402 and the pulse 404 in FIG. 4.

In another implementation, the delta in absolute pulse of the distance from a point B to a point C in pulse 402, and the distance from a point C to pulse D in pulse 404 in FIG. 4 may be determined to obtain accurate data.

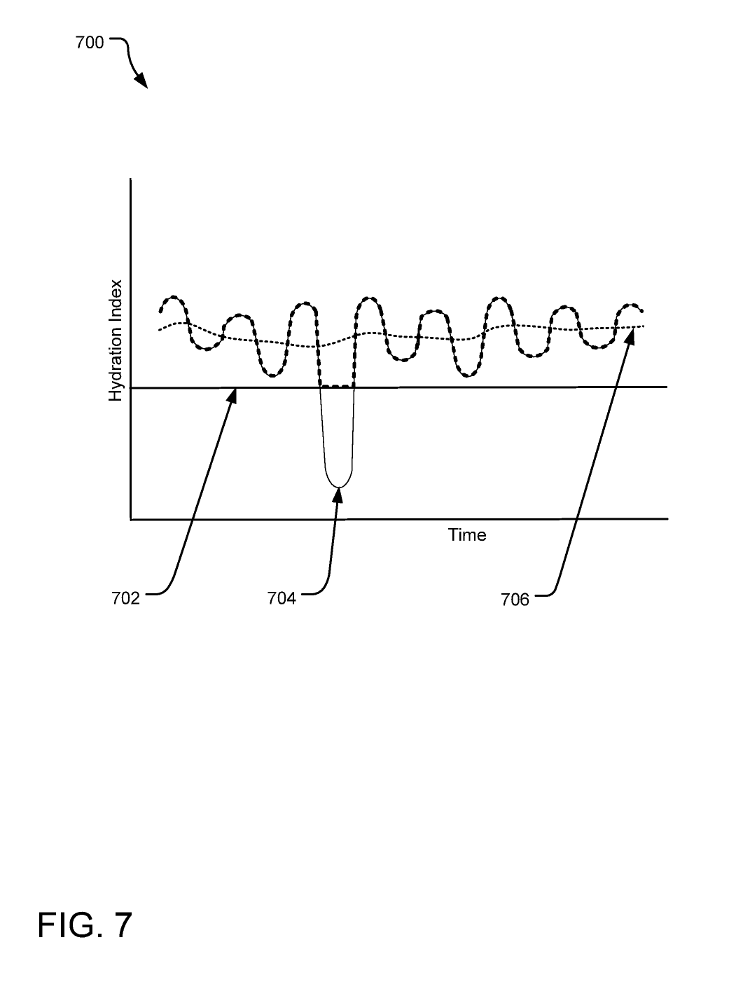

Referring back to FIG. 5, after receiving and filtering the data in operations 502 and 504, there is a predetermined threshold for acceptable data. As will be discussed further in FIG. 7, parameters may be set in a user profile that if a particular condition occurs, then certain acceptable values may be selected, meeting a predetermined threshold, and further analyzed, or discarded.

If the threshold is not met in a threshold operation 506, then the data is deemed invalid or unacceptable and is discarded in a discarding operation 508. The discarded data will not be used in analysis. In another implementation, the data is simply not used in further calculations.

If the threshold for acceptable data is met in a threshold operation 506, then the data is interpolated into a hydration calculator in an interpolating operation 510. After interpolating the data, hydration metric values are derived and data may be provided for post-processing in an operation 512. The operations 500 may occur iteratively, or for a predetermined time period or threshold.

In one implementation (not shown in FIG. 5), the hydration metric values may be used to refine non-invasive blood pressure calculations. Obtaining accurate measurements of arterial blood pressure by non-invasive methods (in the periphery) can be challenging because volume and flow changes may not be linearly correlated with arterial pressure. It is desirable to transform the peripheral volume signal to arterial pressure. Because hydration changes compliance of the vasculature, identifying a hydration metric by the methods disclosed herein can refine non-invasive blood pressure calculations to account for change in vasculature compliance. For example, the pulse interval between an EKG signal and the pressure pulse at an extremity can be more accurately analyzed.

As provided, the aforementioned schemes are implemented pre-processing. These schemes provide information regarding which pulses should be selected for hydration analysis. Schemes for post-processing are performed after a hydration metric is obtained and before data is displayed. Post-processing schemes can include smoothing algorithms, modeling, and other methods to smooth the hydration metric data results.

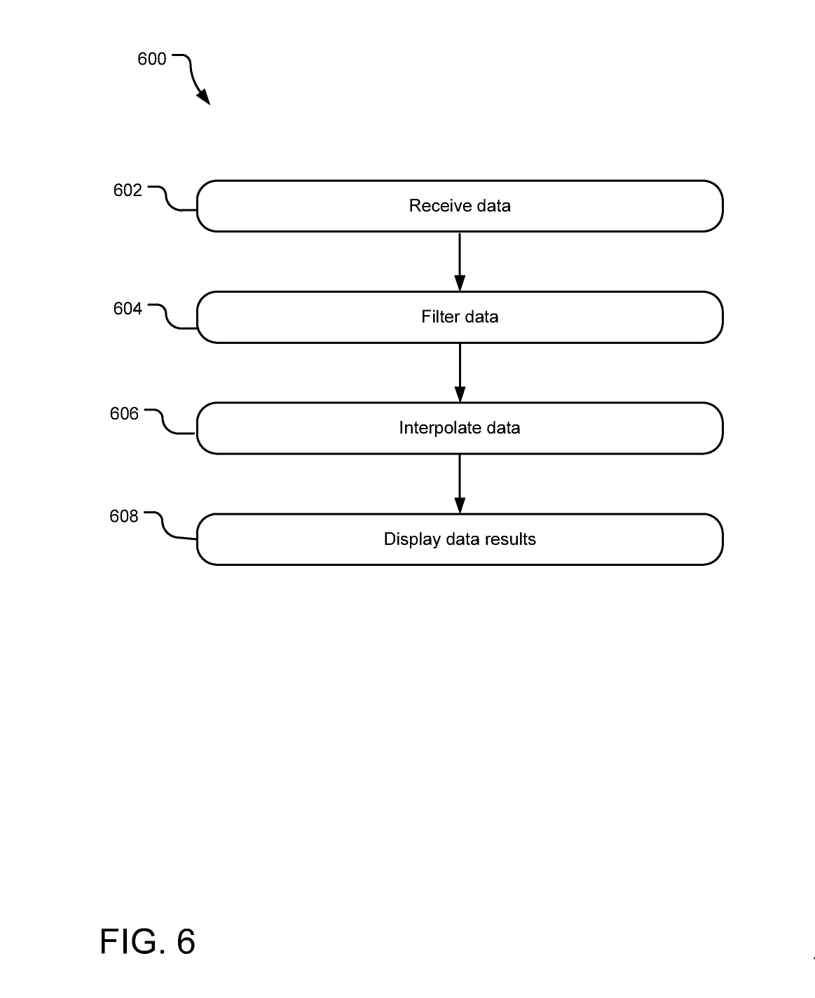

FIG. 6 illustrates a flowchart of example operations 600 for determining result integrity of hydration monitoring data. The hydration metric values are received post-processing in a receiving operation 602. The values are then analyzed for result integrity in an analyzing operation 604. In some implementations, acceptable and unacceptable hydration metric values may be determined using post-processing filter schemes, such as signal smoothing algorithms (e.g., a Savitzky-Golay filter), modeling, and other methods to smooth the hydration metric. For example, if the heart rate is too high or too low, certain hydration metric values may be discarded.

In another implementation, averages may be used for determining result integrity. When taking averages of hydration metric values, if one number is really high, or if the sensor readings are deviating abruptly from the recent average, further analysis may be implemented to determine value integrity. If the number reaches a predetermined number, a threshold may be set, and the data discarded. For example, in a rolling average, if the average value is 90, a value for 73 in the data set may be discarded. This averaging method is illustrated and described in more detail below in FIG. 7.

In another implementation for determining result integrity, to address motion artifact, an accelerometer may be included in the monitor to detect motion. When the accelerometer output signal exceeds predetermined threshold values, instructions in the controller of the monitor will turn off the sensor and the last known good value for the hydration metric is displayed. Once the accelerometer output signal falls below the predetermined threshold, sensing can resume and an updated hydration metric value may be calculated and displayed. After a post-processing filtering scheme in a filtering operation 604, the selected values may be used to determine hydration levels in an interpolating operation 606. The results of the interpolating operation 606 may be displayed on an interface or display unit in a displaying operation 608.

As shown in FIG. 7, a graph 700 depicts an example of filtering of post-processing hydration metric data results versus time. These data results can be also be measured by body weight. If a hydration metric data sample 704 measures outside a predetermined range of values, that data sample 704 may be real or it may be an anomaly. For example, if a user stands up, a data sample may spike or deviate from the other samples. A predetermined absolute or relative cut-off 702 can be implemented wherein if a data sample falls outside a range of other samples (like data sample 704), any value below the cut-off 702 will not be considered as an acceptable hydration metric data sample. Thus, averages of the hydration metric data samples may be taken within a predetermined range and questionable data samples can be discarded (e.g., smoothing line 706).

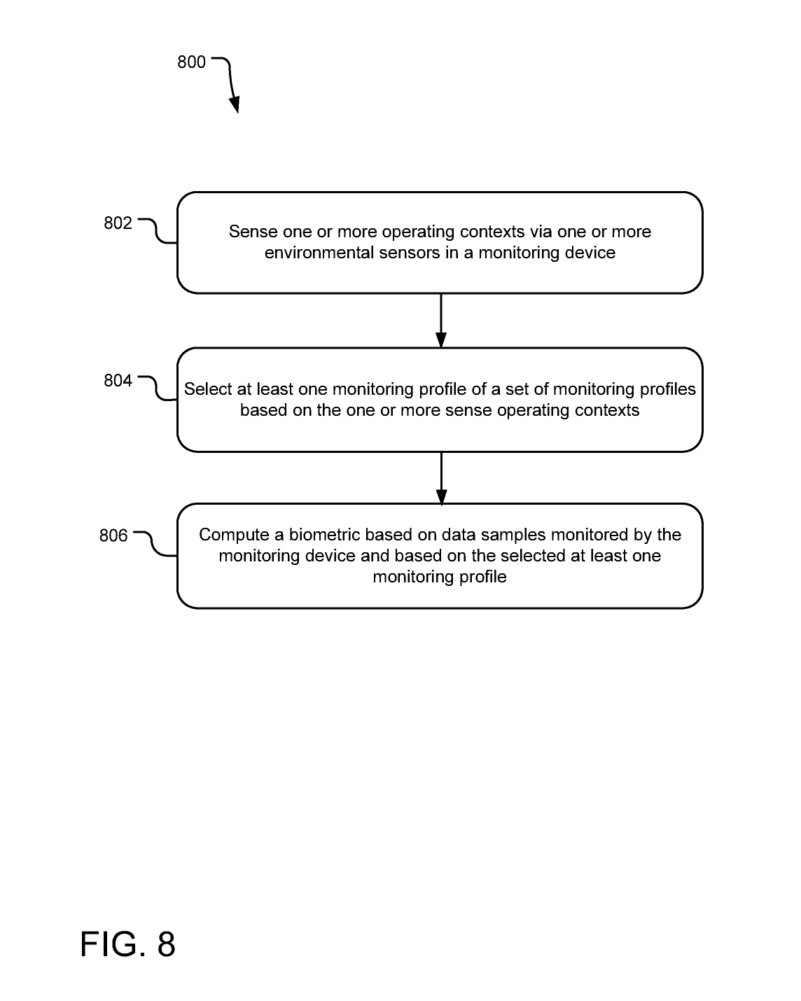

FIG. 8 illustrates example operations 800 for determining a dynamic profile with hydration monitoring data. A sensing operation 802 senses one or more operating contexts. The contexts can be measured by an environmental sensor in a monitoring device.

A selecting operation 804 selects and/or filters the received data based on at least one monitoring profile of a set of monitoring profiles based on the one or more sensed operating contexts, or a non-sensed operating context. The selecting operation 804 uses a variety of profiles, which comprise thresholds, margins, and/or parameters based on the operating contexts, which can include a predetermined range of acceptable data samples. Each monitoring profile can define a data integrity or result integrity condition for the data samples. The data samples selected can be based on each data sample satisfying the data integrity or result integrity condition in the monitoring profile.

Per the profiles, the selecting operation 804 can accept only a subset of acceptable PPG pulse data, while filtering out unacceptable PPG pulse data. For example, based on a sensed environmental condition of altitude, when a user is in high altitude, atmospheric pressure and partial pressure of oxygen can decrease exponentially. Blood pressure and systemic vascular resistance can rise, which can directly effect hydration monitoring calculations. It may be desirable to exclude certain PPG pulse data or to change the hydration monitoring equation in light of such environmental conditions.

In another example, there may be certain ranges of data acceptable based on a particular sensed activity (e.g., when a user is exercising, stationary, or sleeping). In another example, a user may input a profile to include only data samples when the user is running.

Similarly, a certain profile may select and/or filter acceptable PPG pulse data measurements when a certain physiological condition (e.g., heart rate, temperature, and sweat volume) exists. For example, if a user's heart rate measures at certain levels, these data samples may be excluded. In another implementation, performance feedback is determined using a combination of hydration and heart rate, temperature, sweat integrity, and sweat composition parameters. In another implementation, a profile may include parameters specific to medical conditions. For example, if the user is on blood thinning medication and/or has atherosclerosis, which could skew the accuracy of hydration monitoring calculations, certain profiles can accommodate for such conditions or omit data that may be unacceptable.

In another implementation, the selecting operation comprises selecting at least one monitoring profile of the set of monitoring profiles based on a non-sensed condition (e.g., demographic information).

The selecting operation 804 may also select and/or filter PPG pulse data with an adaptive ability to optimize based on input. If the selecting operation 804 senses a change in one or more operating contexts, it may select at least one different monitoring profile of the set of monitoring profiles based on the sensed changes in the one or more operating contexts and compute a new biometric (e.g., a hydration monitoring metric) based on the last least one selected different monitoring profile. As contexts change, the selecting operation 804 may dynamically select new profiles. Changes can include changes environmental conditions (changes in light), changes in sensed activities (changes in movement), changes in physiological conditions (e.g., glucose level or blood pressure), or changes in non-sensed operating contexts (e.g., weight or age). Predetermined ranges in the profiles may be dynamically increased or decreased to accept data under certain sensing operations.

For example, the data selection may include adjusting LED output, sensor gain to compensate for changes in ambient light, or optimizing hydration calculations based on values for heart rate, temperature, and/or accelerometer readings. In one example, there may be selection adjustment depending on whether a user changes position from sitting to standing. During another example selection operation, the selection operation filters data if a profile range of vascular or tissue PPG measurements falls above or below a certain range, and adjusts the range if the physiological condition changes.

In one example, alarms are implemented to provide a user with information with certain condition indicators. For example, overhydration or dehydration alarms can signal wearables, water sources, and other appliances. Such conditions can be tailored to when a user is at rest and/or during a certain activity. In another implementation, profiles could be selected based on different temperatures and enable alarms when the sensed temperature changes.

Once the data is selected and/or filtered per the profile parameters, a biometric (e.g., hydration metric) can be computed in a computing operation 806. The computing operation 806 is based on the PPG pulse data samples monitored by the monitoring device and based on selected monitoring profiles.

The monitoring profiles can define the biometric computation process for the computing operation 806. For example, if the operating contexts change, and a different monitoring profile is selected, the computation process can change. For example, if a user changes a level of activity, or if the user's heart rate or vascular or tissue PPG measurements change, the computation of data samples (e.g., the ratio of determined changes in tissue volume and vascular volume) can change. In some implementations, different monitoring profiles can invert the ratio, change constants and/or multipliers.

In another implementation, after the data results are obtained, a communicating operation communicates a computed biometric via a communications interface. For example, in one implementation, a sports performance index derived from a hydration metric and heart rate analysis may be communicated via a communications interface on a wristlet.

In another implementation, there may also be a calibrating or recalibrating method. For example, a user may put on a monitor and drink water. Analysis of how the user's body responds to water intake may be performed. Predictions may be made regarding what a predetermined threshold of hydration is for that specific user. Over time, such calibrations may adjust and recalibrate according to change. If the device is shared and a new user inputs non-sensed conditions, for example, the new user's demographic information, the device may recalibrate.

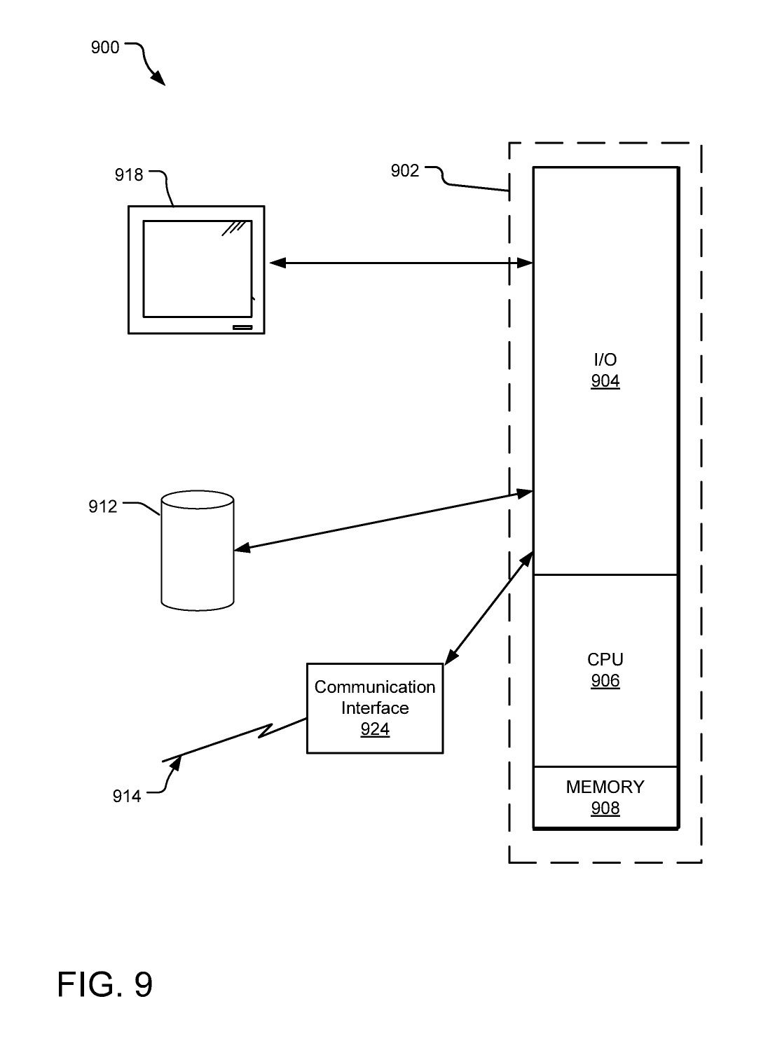

Referring to FIG. 9, a block diagram of a computer system 900 suitable for implementing one or more aspects of a system for receiving and analyzing PPG pulse data and determining a hydration metric is shown. The computer system 900 is capable of executing a computer program product embodied in a tangible computer-readable storage medium to execute a computer process. Data and program files may be input to the computer system 900, which reads the files and executes the programs therein using one or more processors. Some of the elements of a computer system 900 are shown in FIG. 9 wherein a processor 902 is shown having an input/output (I/O) section 904, a Central Processing Unit (CPU) 906, and a memory section 908. There may be one or more processors 902, such that the processor 902 of the computing system 900 comprises a single central-processing unit 906, or a plurality of processing units. The processors may be single core or multi-core processors. The computing system 900 may be a conventional computer, a distributed computer, or any other type of computer. The described technology is optionally implemented in software loaded in memory 908, a disc storage unit 912, and/or communicated via a wired or wireless network link 914 on a carrier signal (e.g., Ethernet, 3G wireless, 5G wireless, LTE (Long Term Evolution)) thereby transforming the computing system 900 in FIG. 9 to a special purpose machine for implementing the described operations.

The I/O section 904 may be connected to one or more user-interface devices (e.g., a keyboard, a touch-screen display unit 918, etc.) or a disc storage unit 912. Computer program products containing mechanisms to effectuate the systems and methods in accordance with the described technology may reside in the memory section 904 or on the storage unit 912 of such a system 900.

A communication interface 924 is capable of connecting the computer system 900 to an enterprise network via the network link 914, through which the computer system can receive instructions and data embodied in a carrier wave. When used in a local area networking (LAN) environment, the computing system 900 is connected (by wired connection or wirelessly) to a local network through the communication interface 924, which is one type of communications device. When used in a wide-area-networking (WAN) environment, the computing system 900 typically includes a modem, a network adapter, or any other type of communications device for establishing communications over the wide area network. In a networked environment, program modules depicted relative to the computing system 900 or portions thereof, may be stored in a remote memory storage device. It is appreciated that the network connections shown are examples of communications devices for and other means of establishing a communications link between the computers may be used.

In an example implementation, a user interface software module, result integrity module, a data integrity module, and other modules may be embodied by instructions stored in memory 908 and/or the storage unit 912 and executed by the processor 902. Further, local computing systems, remote data sources and/or services, and other associated logic represent firmware, hardware, and/or software, which may be configured to assist in obtaining hydration measurements. A hydration monitoring system may be implemented using a general purpose computer (located outside or inside the monitoring device) and specialized software (such as a server executing service software), a special purpose computing system and specialized software (such as a mobile device or network appliance executing service software), or other computing configurations. In addition, PPG pulse data samples, profiles, hydration metric data results, and system optimization parameters may be stored in the memory 908 and/or the storage unit 912 and executed by the processor 902.

It should be understood that the hydration monitoring system may be implemented in software executing on a stand-alone computer system, whether connected to a hydration monitor device or not. In yet another implementation, the hydration monitoring system may be integrated into a device (e.g., a wristlet).

The implementations of the invention described herein are implemented as logical steps in one or more computer systems. The logical operations of the present invention are implemented (1) as a sequence of processor-implemented steps executed in one or more computer systems and (2) as interconnected machine or circuit modules within one or more computer systems. The implementation is a matter of choice, dependent on the performance requirements of the computer system implementing the invention. Accordingly, the logical operations making up the implementations of the invention described herein are referred to variously as operations, steps, objects, or modules. Furthermore, it should be understood that logical operations may be performed in any order, adding and omitting as desired, unless explicitly claimed otherwise or a specific order is inherently necessitated by the claim language.

Data storage and/or memory may be embodied by various types of storage, such as hard disk media, a storage array containing multiple storage devices, optical media, solid-state drive technology, ROM, RAM, and other technology. The operations may be implemented in firmware, software, hard-wired circuitry, gate array technology and other technologies, whether executed or assisted by a microprocessor, a microprocessor core, a microcontroller, special purpose circuitry, or other processing technologies. It should be understood that a write controller, a storage controller, data write circuitry, data read and recovery circuitry, a sorting module, and other functional modules of a data storage system may include or work in concert with a processor for processing processor-readable instructions for performing a system-implemented process.

For purposes of this description and meaning of the claims, the term “memory” (e.g., memory 320, memory 908) means a tangible data storage device, including non-volatile memories (such as flash memory and the like) and volatile memories (such as dynamic random access memory and the like). The computer instructions either permanently or temporarily reside in the memory, along with other information such as data, virtual mappings, operating systems, applications, and the like that are accessed by a computer processor to perform the desired functionality. The term “memory” expressly does not include a transitory medium such as a carrier signal, but the computer instructions can be transferred to the memory wirelessly.

The above specification, examples, and data provide a complete description of the structure and use of exemplary implementations of the invention. Since many implementations of the invention can be made without departing from the spirit and scope of the invention, the invention resides in the claims hereinafter appended. Furthermore, structural features of the different implementations may be combined in yet another implementation without departing from the recited claims.

What is claimed is:

1. A method comprising:
sensing one or more operating contexts via one or more environmental sensors in a monitoring device;
selecting at least one monitoring profile of a set of monitoring profiles based on the one or more sensed operating contexts; and
computing a biometric based on data samples monitored by the monitoring device and based on the selected at least one monitoring profile.

2. The method of claim 1, wherein the one or more sensed operating contexts include at least one of a sensed activity, an environmental condition, or a physiological condition.

3. The method of claim 1, wherein the selecting operation comprises selecting at least one monitoring profile of the set of monitoring profiles based on a non-sensed condition.

4. The method of claim 1, further comprising:
sensing a change in the one or more operating contexts;
selecting at least one different monitoring profile of the set of monitoring profiles based on the sensed changes in the one or more operating contexts; and
computing a new biometric based on the at least one selected different monitoring profile.

5. The method of claim 1, wherein the at least one monitoring profile defines a biometric computation process for the computing operation.

6. The method of claim 5, wherein the biometric computation process comprises determining changes in tissue volume and changes in vascular volume within body tissue of a subject.

7. The method of claim 6, wherein the computing operation comprises computing the biometric as a ratio of the determined changes in tissue volume to the determined changes in vascular volume.

8. The method of claim 6, wherein the computing operation comprises computing the biometric as a ratio of the determined changes in vascular volume to the determined changes in tissue volume.

9. The method of claim 1, wherein the at least one monitoring profile of the set of monitoring profiles defines a data integrity condition for the data samples.

10. The method of claim 9, wherein the data samples used in computing the biometric are selected based on each selected data sample satisfying the data integrity condition.

11. The method of claim 10, wherein the data samples are taken from a plethysmographic (PPG) waveform, and the data integrity condition defines one or more PPG waveform characteristics of the data samples for use in the computing operation.

12. The method of claim 1, wherein the at least one monitoring profile of the set of monitoring profiles defines a result integrity condition for the data samples.

13. The method of claim 11, wherein result integrity condition includes a smoothing algorithm.

14. The method of claim 1, wherein the at least one monitoring profile comprises a predetermined range of acceptable data samples.

15. The method of claim 14, wherein the predetermined range of acceptable data samples dynamically adjusts based on a change in at least one of the sensed operating contexts.

16. The method of claim 15, wherein the predetermined range of acceptable data samples increases based on the change in the one or more sensed operating contexts.

17. The method of claim 15, wherein the predetermined range of acceptable data samples decreases based on the change in the one or more sensed operating contexts.

18. The method of claim 1, further comprising enabling an alarm when the sensed operating contexts change.

19. A system comprising:
a biometric monitoring processor configured to sense one or more operating contexts via one or more environmental sensors in a monitoring device, select at least one monitoring profile of a set of monitoring profiles based on the one or more sensed operating contexts, and compute a biometric based on data samples monitored by the monitoring device and based on the selected at least one monitoring profile; and
a memory storing the set of monitoring profiles.

20. The system of claim 19, wherein the one or more environmental sensors include at least one of a light sensor, a gyroscope, a temperature monitor, an accelerometer, or an electrode in a monitoring device.

21. The system of claim 19, wherein the operating contexts include at least one of a sensed activity, an environmental condition, or a physiological condition.

22. The system of claim 19, further comprising a communications interface configured to communicate the computed biometric.

23. One or more tangible computer-readable storage media encoding computer-executable instructions for executing on a computer system a computer process for computing a biometric, the computer process comprising:
sensing one or more operating contexts via one or more environmental sensors in a monitoring device;
selecting at least one monitoring profile of a set of monitoring profiles based on the one or more sensed operating contexts; and
computing a biometric based on data samples monitored by the monitoring device and based on the selected at least one monitoring profile.

24. The one or more tangible computer-readable storage media of claim 23, further comprising:
sensing a change in the one or more operating contexts;
selecting at least one different monitoring profile of the set of monitoring profiles based on the sensed changes in the one or more operating contexts; and
computing a new biometric based on the at least one selected different monitoring profile.

25. The one or more tangible computer-readable storage media of claim 24, wherein the at least one selected monitoring profile defines a hydration metric computation process for the computing operation.

26. The one or more tangible computer-readable storage media of claim 24, wherein the hydration metric computation process further comprises computing a hydration metric as a ratio of the determined changes in tissue volume to the determined changes in vascular volume.

27. The one or more tangible computer-readable storage media of claim 25, wherein the hydration metric computation process further comprises measuring photoplethysmographic (PPG) waveforms representative of the changes in tissue volume and changes in vascular volume within the body tissue of the subject.

28. The system of claim 26, wherein the hydration metric computation process further comprises computing a tissue pressure area of the PPG waveform indicative of changes in tissue volume and a vessel pressure area of the PPG waveform indicative of changes in vascular volume.

29. The system of claim 27, wherein the hydration metric computation process further comprises further comprises computing the hydration metric as a ratio of the tissue pressure area to the vessel pressure area.

30. The system of claim 28, wherein the hydration metric computation process further comprises computing the hydration metric as a ratio of the vessel pressure area to the tissue pressure area.
